(19) United States
(12) Patent Application Publication
(10) Pub. No.: US 2014/0363489 A1
(43) Pub. Date: Dec. 11, 2014

(54) TREATMENT OF EYE DISEASE

(71) Applicant: Panacea Pharmaceuticals, Gaithersburg, MD (US)

(72) Inventors: Hossein A. Ghanbari, Potomac, MD (US); Zhi-Gang Jiang, Gaithersburg, MD (US)

(21) Appl. No.: 13/912,681

(22) Filed: Jun. 7, 2013

(51) Int. Cl. C07D 213/73

(52) U.S. Cl. CPC C07D 213/73
USPC 424/450; 514/353

(57) ABSTRACT

The present invention provides methods and compositions for treating retinal degeneration. One embodiment of the present invention is directed to a method of treating retinal degeneration by administering to a patient in need at least one thiosemicarbazone compound.

### BACKGROUND OF THE INVENTION

Retinitis pigmentosa (RP) is an inherited, late-onset and slowly progressing retinal neurodegenerative disease affecting only rod photoreceptor neurons in the early stage of disease. RP eventually leads to total blindness. RP is characterized by nyctalopia, ring scotoma, and bone-spicule pigmentation of the retina. RP affects ˜1 out of 4000 individuals worldwide. Currently, there are no successful treatments for patients with RP.

As a possible molecular etiology of RP, retina-specific gene defects are most likely involved. With a complex genetic profile leading to a heterogeneous molecular etiology, RP is a challenging therapeutic target. Although >100 RP-inducing mutations have been identified, gene therapy is still in the early stages of development. Several efforts for RP treatment are under development. One approach is to deliver neurotrophic factor into eye. Ciliary neurotrophic factor (CNTF) has demonstrated therapeutic potential. However, due to its relatively short half-life in vivo (120-400 min), as well as the associated costs of protein purification, intravitreal injection of CNTF is both prohibitively expensive and inefficient. Neurotech Pharmaceuticals has developed a unique solution for chronic delivery of CNTF by using an inventive tissue engineering strategy, called Encapsulated Cell Technology (ECT), and a CNTF-secreting cell line. Neurotech is testing their device in a Phase I clinical trial in patients with RP. Cell therapy could be an alternative strategy for RP therapy. Subretinal transplantation of stem cell-differentiated retinal pigment epithelial (RPE) cells into Royal College of Surgeons (RCS) rats, a model showing a progressive photoreceptor loss during the first 3 months after birth, rescued the photoreceptor cells directly over the grafted RPE cells from degeneration. Another potential treatment for retinal degeneration is gene therapy. RPE65 is an isomerohydrolase expressed in retinal pigment epithelium, and is critical for the regeneration of the visual pigment necessary for both rod and cone-mediated vision. Mutations in human RPE65 cause Leber's congenital amaurosis (LCA) and other forms of autosomal recessive retinitis pigmentosa which are associated with early-onset blindness. At least, three clinical trials are currently underway for the treatment of LCA using modified adeno-associated virus (AAV) vectors carrying the RPE65 cDNA and have reported positive preliminary results (Cai et al., 2009).

So far, no effective therapy has been found for RP. Little has been identified in terms of intracellular mechanisms leading to retinal photoreceptor cell death at post-translational levels. Recently, extracellular free calcium influx and intracellular reactive oxygen species (ROS) accumulation have been demonstrated to play important roles in the degeneration process. To find the common toxic pathway(s), retinal function and morphology, retinoid level, rhodopsin regeneration, rhodopsin phosphorylation and dephosphorylation, and cytosolic cGMP levels were examined in several animal RP models with different causes. These models include RCS rats with a deficit of retinal pigment epithelium (RPE) function caused by rhodopsin mutation, P23H rats, S334ter rats, photo stress rats, retinal degeneration (rd) mice with a deficit of phosphodiesterase (PDE) function, and cancer-associated retinopathy (CAR) model rats with a deficit of recoverin-dependent photoreceptor adaptation function. In these models, lack of regulation of photoreceptor adaptation processes caused by an imbalance of rhodopsin phosphorylation and dephosphorylation was found causing retinal dysfunction leading to photoreceptor cell death. As possible candidate drugs for normalizing these retinal dysfunctions and ending further retinal degeneration, nilvadipine, a calcium channel blocker, retinoid derivatives, and anthocyanine were chosen and tested to determine their effect on the above animal models with retinal degeneration. Nilvadipine showed beneficial effects against retinal degeneration in all models tested, but retinoid derivatives and anthocyanine showed these beneficial effects in only some models (Ohguro, 2008). Thus, intracellular free calcium accumulation is likely a key, common step for retinal degeneration in RP with different gene defects.

As there are no proven treatments, there is a need for new methods to properly treat RP. The present invention provides just such a method.

### SUMMARY OF THE INVENTION

The present invention is directed to a method of treating retinal degeneration.

One embodiment of the present invention is directed to a method of treating retinal degeneration by administering to a patient in need at least one thiosemicarbazone compound.

Another embodiment of the present invention is directed to a method of treating retinal degeneration in RP by administering to a patient in need a composition comprising 3-aminopyridine-2-carboxaldehyde thiosemicarbazone, or an analogue thereof.

Another embodiment of the present invention is directed to a method of treating retinal degeneration in RP by administering to a patient in need a composition comprising 3-aminopyridine-2-carboxaldehyde thiosemicarbazone where the step of administering is intraocular, intravitreal, intravenous, intraperitoneal, subcutaneous, intramuscular, topical, transdermal or oral.

The present invention further encompasses methods of treating retinal degeneration in RP by administering a composition comprising a compound of Formula I, or an analogue thereof:

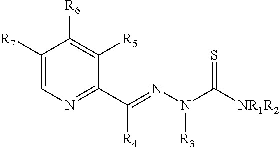

Wherein R, R₁, R₂, R₃, and R₄ are independently selected from the group consisting of hydrogen, C1-8alkyl, C2-8alkenyl, C2-8alkynyl, C3-8cycloalkyl, C1-8haloalkyl, C6-10aryl, amino-C1-8alkyl, hydroxy-C1-8alkyl, C1-8alkoxye-C1-8alkyl, and C1-C8alkanoyl, or NR₁R₂ taken in combination form a 3 to 7 member ring which may comprise 0, 1, or 2 additional ring heteroatoms selected from N, O, and S; R₆ is hydrogen, hydroxy, amino, or C1-8alkyl; R₅ and R₇ are independently selected from the group consisting of hydrogen, halide, hydroxy, thiol, amino, hydroxyamino, mono-C1-8alkylamino, di(C1-8alkyl)amino, C1-8alkoxy, C1-8alkyl, C1-8alkenyl, and C2-8alkynyl.

The present invention further encompasses methods of treating retinal degeneration in RP by administering a composition comprising a compound of Formula II, or an analogue thereof:

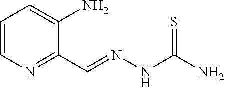 [SMILES: NC(=S)N/N=C/c1ncccc1N]

### BRIEF DESCRIPTION OF THE FIGURE

FIG. 1 shows one embodiment of the present invention where PAN-811 prevents loss of photoreceptor neurons in rd1 mice

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments such as retinal degeneration by artery or vein occlusion, diabetic retinoplasty, retrolental fibroplasia, retinopathy of prematurity, glaucoma, macular degeneration, choroideremia, juvenile retinoschisis, Stargardt disease, Usher disease, and Leber's congenital amaurosis. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is directed to a method for the treatment of retinal degeneration in RP comprising the step of administering to a patient a composition comprising a thiosemicarbazone compound. The means for synthesis of thiosemicarbazone compounds useful in the methods of the invention are well known in the art. Such synthetic schemes are described in U.S. Pat. Nos. 5,281,715; 5,767,134; 4,447,427; 5,869,676 and 5,721,259; all of which are incorporated herein by reference in their entirety.

One such thiosemicarbazone compound, PAN-811, also known as 3-aminopyridine-2-carboxaldehyde thiosemicarbazone, has previously shown a potent neuroprotective activity in ischemic stroke model, with capabilities of reducing intracellular calcium accumulation and chelation of free calcium (Jiang et al., 2006). Based on its role as a potent free calcium chelating agent, we hypothesized that PAN-811 is a therapeutic agent for retinal degeneration in RP. The chemical structures of PAN-811's analogues are shown in U.S. Pat. No 7,456,179, and patent applications of 20090275587, 20060194810 and 20060160826 each of which are hereby incorporated by reference.

The pharmaceutical compositions required by the present invention typically comprise a compound useful in the methods of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intraocular, intravitreal, intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes. Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., one or more thiosemicarbazones) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A compound of the invention can be formulated into a pharmaceutical composition wherein the compound is the only active agent therein. Alternatively, the pharmaceutical composition can contain additional active agents. For example, two or more compounds of the invention may be used in combination.

The effect of PAN-811 was examined in an ex vivo model, in which retinas were isolated from rd1 mouse at PN7 and treated in culture with 0, 2 and 10 μM PAN-811 for 21 days (equal to PN28). The results demonstrated that the rd1 mouse retina has only a thin photoreceptor layer by 21 days. In contrast, PAN-811 treatment (at 10 μM) prevented the loss of photoreceptor neurons in the retina, resulting in a photoreceptor layer of normal thickness.

The one model of RP study is rd1 mouse in which, like in RP patient, the gene for β-phospodiesterase is spontaneously mutated in rod photoreceptor neurons results in the rod cell loss. The apoptosis starts at postnatal day (PN) 9 to 10, peaks at PN13 and is completed at PN21.

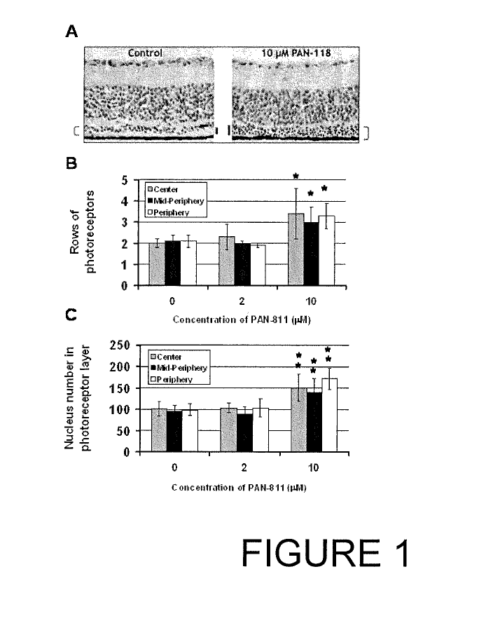

To test the effect PAN-811, retinas from rd1 (C3H strain) mice at PN7 were cultured in the presence of 0 (n=6), 2 (n=6) and 10 (n=7) μM PAN-811. After 21 days in vitro (equal to PN28), the hematoxylin/eosin stained retinal section was analyzed at five positions; the very center of the retina plus at two positions on each side of this, approximately 200 μm apart, and referred to as mid-periphery and periphery, respectively. The retina from the rd1 mouse commonly carried only a thin layer of photoreceptor neurons (FIG. 1A—photo on left). In contrast, this layer was thicker for the retina received a continuous treatment with 10 μM PAN-811 (FIG. 1B—photo on right). Quantitatively, a control group has two layers of photoreceptor neurons at center, mid-periphery or pheriphery (FIG. 1B). PAN-811 at a concentration of 10 μM, but not 2 μM, increased the row numbers from 2 to 3 (P<0.05) at all measured areas.

Nucleus numbers for photoreceptor neurons that locate in center (n=7), mid-periphery (n=6) and periphery (n=6) were counted on photos (within 35 mm length of rode photoreceptor layer after magnification) by ImageJ software (NIH) and the data were expressed as percentage of untreated control. Identical to the row counting, PAN-811 at a concentration of 10 μM, but not 2 μM, increased the nucleus numbers by about 50% (P<0.01 by comparing with untreated control) in all center, mid-periphery and periphery areas (FIG. 1C).

### EXAMPLE 1

Twenty retinas from 10 rd1 mice (C3H strain) were dissected out at PN7 and mounted flat on HA mixed cellulose, 0.45 μm membranes fitted to culture dish inserts (Millipore Corp). The retinas were kept in R16 medium for 21 days. The retinas were treated with vehicle (n=6), 2 μM (n=6) and 10 μM PAN-811 (n=7) with medium replacement every second day. At 21 d.i.v. (equal to PN28), the preparations were fixed in buffered 4% paraformaldehyde and subsequently cut into 10 μm sections with a cryotome and stained by hematoxylin/eosin. The sections were analyzed and photographed under a microscope, blindly with respect to origin The rows of photoreceptors in the outer nuclear layer of the retina were quantitatively evaluated in a total of at least 30 sections for each preparation (4 slides from each preparation and 8 sections from each slide). The value of photoreceptor rows in each retinal section was analyzed at five positions: the center of the retina plus at two positions on each side of this, and another two approximately 200 μm apart, and referred to as mid-periphery and periphery, respectively. The values from both sides yield an average for Center, Mid-periphery, or Periphery. The retina were photographed with Zeiss Axiophot, equipped with a Zeiss Axiocam and nucleus numbers of photoreceptor neurons in rod photoreceptor region were analyzed along a length of 275 μm with ImageJ software (NIH) within threshold range of 82-111 (unless special differentiation needed).

The recorded values of rows of photoreceptors from rd1 mouse retinas were analyzed statistically using a multigroup ANOVA. Differences were considered to be significant if P<0.05. Data were expressed as Means±SD.

As mentioned in Background in Invention, so far, no effective therapy has been found for RP, although neurotrophic factors, gene and cell therapy are under trials for the therapy. Intracellular calcium accumulation has demonstrated a common pathway for RP with different gene deficient. Nilvadipine, an L-type calcium channel blocker, demonstrated a beneficial effect against retinal degeneration in RSC rats. Since extracellular calcium influx not only goes through L-type calcium channel under RP condition, NMDA type of glutamate receptor may be involved as well, an approach in suppression intracellular calcium accumulation will be more effective in regardless the entry paths. Therefore, calcium chelation by PAN-811 represents a novel method for RP therapy.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method for the treatment of retinal degeneration comprising the step of administering to a patient a composition comprising at least one thiosemicarbazone compound, or an analogue thereof.

2. The method of claim 1, wherein the at least one thiosemicarbazone compound comprises 3-aminopyridine-2-carboxaldehyde thiosemicarbazone (PAN-811).

3. The method of claim 2, wherein the step of administering is intraocular, intravitreal, intravenous, intraperitoneal, subcutaneous, intramuscular, topical, transdermal or oral.

4. The method of claim 2, wherein the composition is an injectable and/or infusable solution.

5. The method of claim 2, wherein the composition is formulated as a micro emulsion.

6. The method of claim 2, wherein the composition is formulated as a liposome.

7. A method for the treatment of retinal degeneration comprising administering to a patient a composition comprising at least one thiosemicarbazone compound (Formula I), or an analogue thereof:

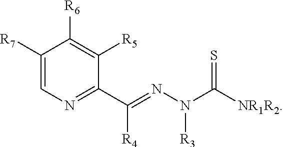

8. The method of claim 7, wherein the at least one thiosemicarbazone compound comprises the compound of Formula II, or an analogue thereof:

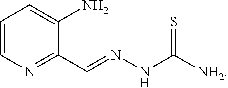 [SMILES: NC(=S)N/N=C/c1ncccc1N]

9. The method of claim 7, wherein the step of administering is intravenous, intraperitoneal, subcutaneous, intramuscular, topical, transdermal or oral.

10. The method of claim 7, wherein the composition is an injectable and/or infusible solution.

11. The method of claim 7, wherein the composition is formulated as a micro emulsion.

12. The method of claim 7, wherein the composition is formulated as a liposome.
